(19) United States
(12) Patent Application Publication
(10) Pub. No.: US 2007/0276467 A1
(43) Pub. Date: Nov. 29, 2007

(54) Device for regulating blood flow

(76) Inventor: Menno Kalmann, Elspeet (NL)

Correspondence Address: Neil D. Gershon, 1011 High Ridge Road, Stamford, CT, 06905, US

(21) Appl. No.: 11/801,489

(22) Filed: May 10, 2007

Related U.S. Application Data
(60) Provisional application No. 60/808,406, filed on May 25, 2006.
(60) Provisional application No. 60/809,483, filed on May 31, 2006.

(51) Int. Cl. A61F 2/06

(52) U.S. Cl. 623/1.24; 623/2.17; 623/1.16

(57) ABSTRACT

An elongated frame dimensioned and configured for implantation in a blood vessel. The frame has two axially spaced apart ring portions, which may be in the form of an expandable crown or a similar structure. At least one linking member connects the two ring portions to one another to form an integral structure. A valve membrane is supported between the axially spaced apart ring portions of the frame. The valve membrane is adapted and configured for movement between a first position in which blood flow through the frame is permitted and a second position in which blood flow through the frame is inhibited.

This application claims priority from provisional application Ser. Nos. 60/808,406, filed May 25, 2006 and 60/809,483, filed May 31, 2006, the entire contents of which are incorporated herein by reference.

### BACKGROUND OF THE INVENTION

1. Field of the Invention

The subject invention is directed to a device for regulating blood flow in the venous system, and more particularly, to an implantable valve device for regulating the flow of blood through a blood vessel.

2. Description of Related Art

The blood system, and in particular the venous blood system of the legs and arms is provided with valves that are uniquely located in a manner so as to ensure that blood will not flow back upstream in the direction from which it has been pumped from the heart. In the arms and legs, there is a deep venous system and a surface venous system. Due to various causes, thrombosis can occur in the deep venous system. Blood thinning can alleviate this problem. However, valves do not effectively close and often leak when the blood in thinned. This can cause increased venous blood pressure in the direction of the ankles, which can lead to a variety of problems including varicose veins and open leg. Complaints of this type are wide spread among those who spend prolonged periods of time in a standing position, for instance, surgeons.

The surface venous system of the leg is relatively weaker than the deep venous system, and it has the tendency to spontaneously widen. This widening prevents the valves from functioning effectively and can lead to varicose veins, which are both unattractive and painful. Major surgery is often required to treat these blood vessel problems. For example, varicose veins are treated by either closing off the vein, which leads to a reduced blood flow capacity and increased pressure on surrounding blood vessels to ensure blood supply, or by completely removing the varicose veins, which leads to the same problem. The subject invention is directed to a device for obviating problems of this type.

### SUMMARY OF THE INVENTION

More particularly, the subject invention is directed to a new and useful implantable valving device for mechanically regulating blood flow through a blood vessel. The device includes, among other things, an elongated frame dimensioned and configured for implantation in a blood vessel. In one aspect, the frame has two axially spaced apart ring portions, which may be in the form of an expandable crown or a similar structure and at least one linking member connects the two ring portions to one another to form an integral structure. A valve membrane is supported between the axially spaced apart ring portions of the frame. The valve membrane is adapted and configured for movement between a first position in which blood flow through the frame is permitted and a second position in which blood flow through the frame is inhibited.

Preferably, the frame includes a plurality of peripherally spaced apart linking members, and it is formed at least in part from a shape memory alloy material. The valve membrane is preferably formed at least in part from a thin non-expanded PTFE membrane or a similar material and may be coated at least in part with an anti-clotting agent.

The subject invention also provides an implantable device for regulating blood flow through a blood vessel comprising an elongated frame, dimensioned and configured to be implanted in a blood vessel, having first and second axially spaced apart frame portions. A valve membrane is supported between the axially spaced apart frame portions and adapted for movement between a first position in which blood flow through the frame is permitted and a second position in which blood flow through the frame is inhibited.

In one aspect, the membrane includes a first body portion and a second narrower neck portion, wherein the first membrane portion is attached to the first frame portion and the second membrane portion is attached to the second frame portion. Preferably, the narrower neck portion has a length at least equal to the radius of the second frame portion. In one embodiment, the first and second frame portions are joined by a linking member extending from opposing sides of the frame portions. In one embodiment, the body portion and neck portion of the membrane are attached to their respective frame portions 180 degrees apart.

Preferably, the first and second frame portions are ring members. The ring members can have a wavy configuration.

In another aspect, the membrane has a curved surface and further has first and second attachment regions attached to the first frame portion and a third attachment region attached to the second frame portion. In one embodiment, the first frame portion and the second frame portions are positioned at an angle to the longitudinal axis of the vessel. The frame preferably includes a linking member joining the first and second frame portions. The first and second frame portions can be positioned at an angle to the linking member.

These and other features of the subject invention will become more readily apparent to those having ordinary skill in the art from the following enabling description of the invention taken in conjunction with the drawings appended hereto.

### BRIEF DESCRIPTION OF THE DRAWINGS

So that those skilled in the art to which the subject invention appertains will readily understand how to make and use the apparatus of subject invention without undue experimentation, preferred embodiments thereof will be described in detail hereinbelow with reference to certain figures, wherein:

FIG. 1 is a perspective view of the frame of a flow-regulating device constructed in accordance with a preferred embodiment of the subject invention;

FIG. 2 is a plan view of a membrane employed with the frame of FIG. 1;

FIG. 3 is a schematic perspective view of the flow-regulating device of the subject invention, wherein the frame is simplified to illustrate the position of the membrane to permit blood flow through a vessel;

FIG. 4 is a schematic perspective view of the flow-regulating device of the subject invention as in FIG. 3, with the membrane oriented to block the flow of blood through a vessel;

FIG. 5 is a perspective view of another flow-regulating device constructed in accordance with another preferred embodiment of the subject invention with the membrane oriented to block blood flow through a vessel;

FIG. 6 is a perspective view as in FIG. 5, with the membrane oriented to permit blood flow through a vessel;

FIG. 7 is a perspective view of yet another flow-regulating device constructed in accordance with another embodiment of the subject invention with the frame in a closed condition;

FIG. 8 is a perspective view of the device shown in FIG. 7, with the frame expanded into an open position;

FIG. 9 is a perspective view of the device of FIG. 7, shown with a balloon catheter prior to deployment in a blood vessel; and

FIG. 10 is a perspective view of the device of FIG. 7, shown with a balloon catheter inflated to expand the device into a deployed condition within a blood vessel.

### ENABLING DESCRIPTION OF PREFERRED EMBODIMENTS

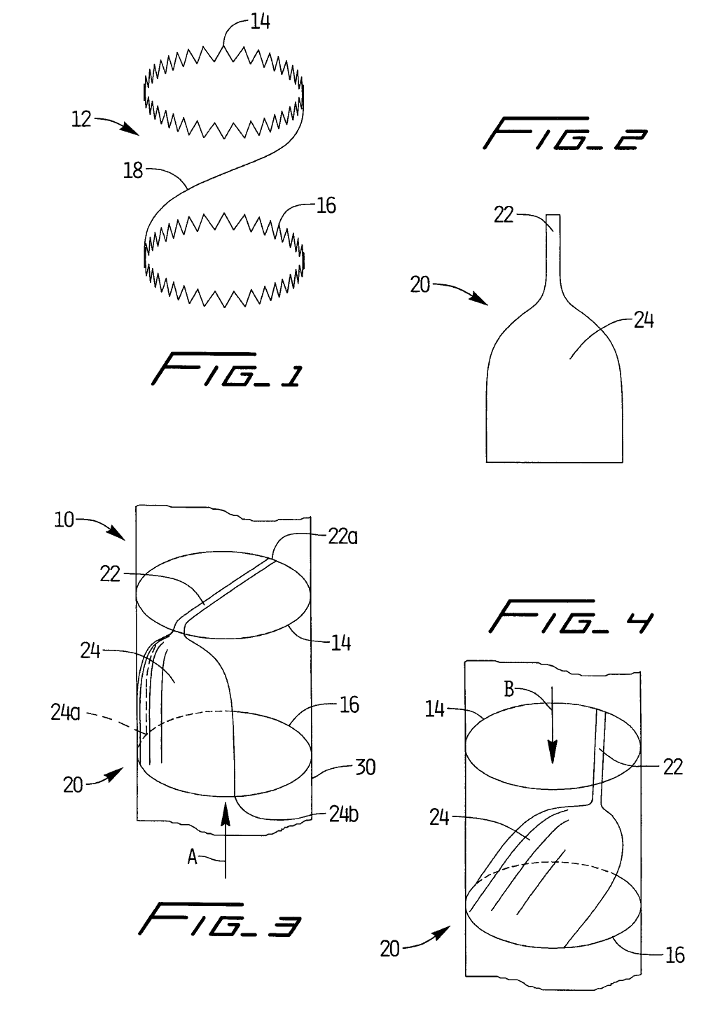

Referring now to the drawings wherein like reference numerals, there is illustrated in FIG. 1, a flow regulating device constructed in accordance with a preferred embodiment of the subject invention, and designated generally by reference numeral 10. Regulating device 10 includes an elongated frame 12 that consists of upper and lower crown-shaped or wave-like rings 14 and 16. That is, each ring has connecting V-shapes as shown. These rings 14, 16 are preferably larger in diameter than the host vein in their expanded placement configuration, to ensure that the device remains in a desired position and orientation after implantation. For example, the diameter of the rings may be 1.25% larger than the diameter of the intended host vein.

Rings 14 and 16 are connected to one another by at least one connective or linking member in the form of a bar or wire 18. For ease of illustration, only one connective wire 18 is shown in FIG. 1. Bar or wire 18 is curved and adapted and configured to follow the circumference of the host vessel. Preferably, the bar or wire 18 is attached to the opposed rings 14 and 16 of frame 12 at locations that are about 180° apart from one another, as shown. This gives frame 12 an inherent flexibility and enables it to move with the natural movements (e.g., peristaltic) of the vein.

Due to the crown-shape of the rings 14 and 16, the device 10 can be reduced to approximately ⅕ of the final implanted diameter and could be introduced into a blood vessel through a relatively small delivery device. For example, a device having a working diameter of 6 F to 8 F could be used.

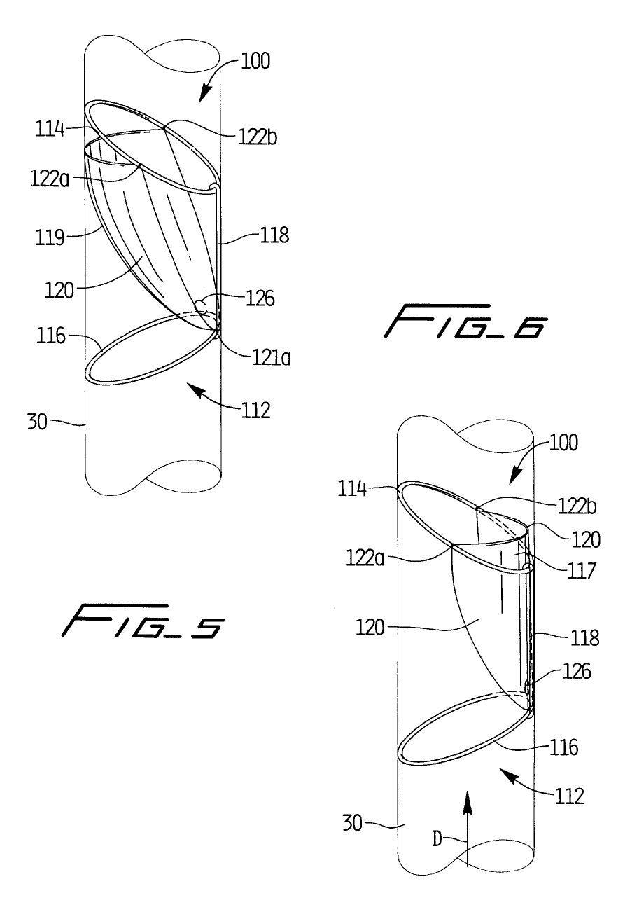

As shown in FIGS. 3 and 4, wherein the connective rod(s) 18 are not shown and the rings 14, 16 are simplified for ease of illustration, device 10 includes a valve member 20 that is operatively associated with frame 12 for regulating the flow of blood through a vessel by moving between open and closed positions. Valve member 20 is formed from a sheet of ultra thin membrane material such as a non-expandable PTFE material or the like. It is envisioned that the membrane could be bonded or otherwise coated with an anti-clotting or anti-coagulant agent such as Heparin.

As best seen in FIG. 2, valve membrane 20 has a narrow elongated neck portion 22 for attachment to the upper ring 14 of frame 12 and a wide body portion 24 for attachment to the lower ring 16 of frame 12. The narrow neck portion 22 as shown extends across the diameter of the device, and preferably has a length greater than a radius of the device (ring) and slightly less than the diameter of the ring. The attachment locations (22a of neck portion 22 and 24a, 24b of body portion 24) of the membrane 20 on each ring are preferably approximately 180° degrees from one another so that the body portion 24 of the membrane 20 will extend substantially if not entirely across the expanse of frame in the closed position shown in FIG. 4. Note the membrane could be attached to ring 16 along its curved perimeter or attached at specific points, e.g. 24a, 24b, or at additional points.

Referring to FIG. 3, blood flowing through the blood vessel 30 in the downstream direction indicted by arrow “A” will act against the valve membrane 20 in such a manner so as to push the wide body portion 24 of the membrane 20 against the wall of the blood vessel 30. At such a time, blood will flow freely through the frame 12, impeded only incidentally by the narrow neck portion 22 of membrane 20 extending across the device.

Referring to FIG. 4, blood flowing through the blood vessel 30 in the upstream direction indicated by arrow “B” will act against the valve membrane 20 in such a manner so as to push the wide body portion 24 in a direction as shown, substantially if not entirely closing off blood flow through the blood vessel 30. Due to the length of the narrow part 22 of the valve membrane 20, the wide body portion 24 will close at a relatively steep angle (e.g., 30°). This is important because the steeper the closure angle, the less force required to push the valve membrane back to an open position with the natural blood pressure.

Referring now to FIG. 5, to minimize the number and complexity of implantable components, an implantable device 100 is provided that includes a frame 112 having two axially spaced part substantially circular rings 114, 116 and a connecting bar 118, with an ultra thin, generally triangular shaped membrane 120 operatively associated therewith. As shown, each of the rings 114, 116 is positioned at an angle, preferably obtuse as shown, to the connecting bar 118. The lower apex of the triangular membrane 120 is attached to the lower ring portion 116 of frame 112 (attachment region 121a), and the upper apices of the triangular membrane 120 are attached to the upper ring portion 114 of frame 112 at diametrically opposed positions (attachment regions 122a, 122b). Preferably, the upper portion of the membrane 120 is loosely attached to the upper ring portion 114, allowing it to slide down the ring during insertion. The lower portion of the membrane 120 is attached to the lower ring portion 116 in the same general area as the connecting rod 118. The membrane has a curved or convex outer surface 119 in the flow blocking position of FIG. 5 and a curved or convex outer surface 117 facing in the opposite direction (radially) in the blood flow position of FIG. 6 (see arrow D).

It is envisioned that the frame 112 (and frame 12) is made from a shape-memory or super-elastic material such as Nitinol or a similar material, so as to enable the collapse and recovery of the rings during implantation in blood vessel 30. The ultra thin membrane 120 is preferably made from a material such as PTFE, and may be provided with an anti-clotting drug.

It is also envisioned that valve membrane 120 can have a small slit or hole 126 adjacent the lower apex of the membrane near the connection with lower ring portion 116 to allow some of the blood trapped behind to flow back through the membrane. This will reduce the likelihood of clotting.

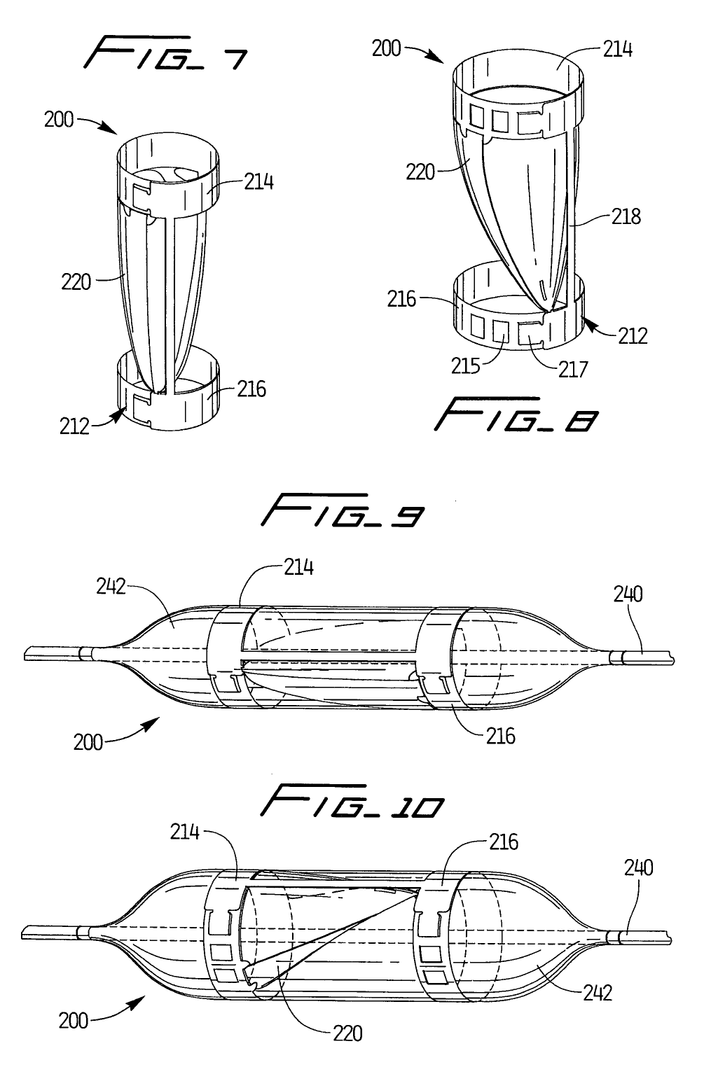

Referring now to FIGS. 7 through 10, there is illustrated another embodiment of the implantable device of the subject invention, which is designated generally by reference numeral 200. Device 200 includes a frame 212 having opposed flexible straps 214, 216 and a connecting structure 218. Straps 214 and 216 are preferably formed from a shape memory material that is normally biased into a coiled or closed configuration, shown for example in FIG. 7. A generally triangular membrane 220 is attached to frame 212 in a manner similar to the way in which membrane 120 is attached to frame 112. The rings 214, 216 of frame 212 are adapted and configured for securement in an expanded or open position, shown in FIG. 8, through the interaction of a locking tangs 217 and apertures 215.

As illustrated in FIGS. 9 and 10, device 200 is implanted in a blood vessel using a balloon catheter 240. More particularly, rings 214 and 216 are moved from a closed position to an expanded position by inflating balloon 242. Upon expansion, to a desired position, tangs 217 engage apertures 215 to lock the rings 214 and 216 in a desired position. The balloon 242 is then deflated and the catheter 240 is removed from the blood vessel so the device 200 can regulate the flow of blood through the vessel, in the manner described previously with respect to device 100.

Although the blood flow-regulating device of the subject invention has been described with respect to preferred embodiments, those skilled in the art will readily appreciate that changes and modifications may be made thereto without departing from the spirit and scope of the subject invention.

What is claimed is:

1. An implantable device for regulating blood flow through a blood vessel, comprising:
a) an elongated frame dimensioned and configured to be implanted in a blood vessel, the frame including axially spaced apart ring portions, and at least one linking member connecting the axially spaced apart ring portions to one another; and
b) a valve membrane supported between the axially spaced apart ring portions of the frame and adapted for movement between a first position in which blood flow through the frame is permitted and a second position in which blood flow through the frame is inhibited.

2. An implantable device as recited in claim 1, wherein the frame includes a plurality of peripherally spaced apart linking members.

3. An implantable device as recited in claim 1, wherein the frame is formed at least in part from a shape memory alloy material.

4. An implantable device as recited in claim 1, wherein the valve membrane is formed at least in part from PTFE.

5. An implantable device as recited in claim 1, wherein the valve membrane is coated at least in part with an anti-clotting agent.

6. An implantable device for regulating blood flow through a blood vessel, comprising:
a) an elongated frame dimensioned and configured to be implanted in a blood vessel, the frame including first and second axially spaced apart frame portions; and
b) a valve membrane supported between the axially spaced apart frame portions and adapted for movement between a first position in which blood flow through the frame is permitted and a second position in which blood flow through the frame is inhibited, the membrane including a first body portion and a second narrower neck portion, the first body portion attached to the first frame portion and the second neck portion attached to the second frame portion.

7. The implantable device of claim 6, wherein the narrower neck portion has a length at least equal to the radius of the second frame portion.

8. The implantable device of claim 6, wherein the first and second frame portions are joined by a linking member, the linking member extending from opposing sides of the frame portions.

9. The implantable device of claim 6, wherein the first and second frame portions are joined by a curved linking member.

10. The implantable device of claim 6, wherein the first and second frame portions each comprise ring-like members.

11. The implantable device of claim 10, wherein each of the rings has a wavy configuration.

12. The implantable device of claim 6, wherein the body portion and the neck portion of the membrane are attached to their respective frame portions approximately 180 degrees apart.

13. An implantable device for regulating blood flow through a blood vessel, comprising:
a) an elongated frame dimensioned and configured to be implanted in a blood vessel, the frame including first and second axially spaced apart frame portions; and
b) a valve membrane supported between the axially spaced apart frame portions and adapted for movement between a first position in which blood flow through the frame is permitted and a second position in which blood flow through the frame is inhibited, the membrane having a curved surface, the membrane further having first and second attachment regions attached to the first frame portion and a third attachment region attached to the second frame portion.

14. The implantable device of claim 13, wherein the first frame portion and second frame portion are positioned at an angle to the longitudinal axis of the vessel.

15. The implantable device of claim 13, wherein the frame includes a linking member joining the first and second frame portions.

16. The implantable device of claim 15, wherein the first frame portion is positioned at an angle to the linking member.

17. The implantable device of claim 16, wherein the second frame portion is positioned at an angle to the linking member, both the first and second frame portion angles being obtuse angles.
